(19) United States
(12) Patent Application Publication
(10) Pub. No.: US 2024/0115185 A1
(43) Pub. Date: Apr. 11, 2024

(54) REGIONAL MOTION DISPLAY AND ASSESSMENT IMAGING

(71) Applicant: Duke University, Durham, NC (US)

(72) Inventors: Olaf T. Von Ramm, Durham, NC (US); John C. Moore, Durham, NC (US)

(21) Appl. No.: 18/376,919

(22) Filed: Oct. 5, 2023

Related U.S. Application Data
(60) Provisional application No. 63/413,749, filed on Oct. 6, 2022.

(51) Int. Cl. A61B 5/346; G06T 5/50; G06T 7/00; G06V 10/25; G06V 10/60; G06V 10/75

(52) U.S. Cl. CPC A61B 5/346; G06T 5/50; G06T 7/0012; G06V 10/25; G06V 10/60; G06V 10/751; G06T 2207/20224; G06T 2207/30048

(57) ABSTRACT

A method of providing a regional motion display includes receiving a set of temporal images of a target area, generating a set of temporal difference images from the set of temporal images and generating a regional motion display from the set of temporal difference images. The regional motion display includes a representative line through the target area along a y-axis and an x-axis representing time. The method further includes displaying the regional motion display. In some cases, generating the regional motion display includes calculating, for each y-axis pixel, an integral of all pixels along a perpendicular or non-perpendicular line to the target area, a contour along the target area, or a surface of the target area in a temporally corresponding difference image of the set of temporal difference images in a difference image and assigning, for each y-axis pixel, the integral of all pixels as a pixel value.

### CROSS-REFERENCE TO RELATED APPLICATIONS

This application claims the benefit of U.S. Provisional Application Ser. No. 63/413,749, filed Oct. 6, 2022.

### BACKGROUND

High-frame-rate ultrasound echocardiography and high-rate repetitive sampling systems such as Doppler imaging have revealed the existence of propagating mechanical waves or propagating events in the myocardium throughout the cardiac cycle. The velocity of these mechanical waves has been determined by speckle tracking and other methods to assess changes in the stiffness of the myocardium during the cardiac cycle. Current techniques are based on gated studies and do not allow continuous imaging during the data acquisition phase. Furthermore, most of the analysis is conducted in the radiofrequency domain, which require high sampling rates and extensive computations. Recognizing these limitations, there is a need for continuous, non-gated data acquisition during normal, high-speed clinical imaging to further study and implement widespread diagnostic solutions to propagating events in the myocardium and other areas in the body.

### BRIEF SUMMARY

Regional motion display and assessment imaging solutions are provided. Advantageously, movement of tissue and/or materials over time is displayed in an easily consumable format. When applied to certain areas of a living entity (e.g., a heart of a human), detailed information including movement of tissue (e.g., a propagating event) over a period of time (e.g., a myocardial tissue over a cardiac cycle) is presented in a format such that physicians can identify abnormalities and disease in a relatively short period of time compared to conventional diagnostic methods. When applied to non-living materials, the movement of the materials is presented in a format such that abnormalities and defects that are otherwise undetectable are readily identifiable.

A method of providing a regional motion display includes receiving a set of temporal images of a target area. The set of temporal images of the target area includes a sequence of images of the target area taken over a period of time. The method further includes generating a set of temporal difference images from the set of temporal images and generating a regional motion display from the set of temporal difference images. The regional motion display includes a representative line through the target area along a y-axis and an x-axis representing time. The method further includes displaying the regional motion display.

In some cases, generating the set of temporal difference images from the set of temporal images includes, for two temporally sequential images of the set of temporal images, calculating an absolute difference in detected brightness value for each pixel and assigning the absolute difference in detected brightness value as a brightness value for a corresponding pixel in a difference image of the set of temporal difference images. In some cases, generating the regional motion display from the set of temporal difference images includes calculating, for each y-axis pixel of the regional motion display, an integral of all pixels along a line perpendicular to the target area, a non-perpendicular line to the target area, a contour along the target area, or a surface of the target area in a temporally corresponding difference image of the set of difference images and assigning, for each y-axis pixel of the regional motion display, the integral of all pixels along the line perpendicular to the target area, the non-perpendicular line to the target area, the contour along the target area, or the surface of the target area as a pixel value for a temporally corresponding portion of the representative line through the target area along the y-axis. In some cases, generating the regional motion display from the set of temporal difference images further includes applying, for each y-axis pixel of the regional motion display, a brightness transfer function to the integral of all pixels along the line perpendicular to the target area, the non-perpendicular line to the target area, the contour along the target area, or the surface of the target area, wherein a result of the brightness transfer function is assigned as the pixel value for the temporally corresponding portion of the representative line through the target area along the y-axis.

In some cases, the method further includes selecting a corresponding region of interest from the target area across the set of temporal difference images. In some of these cases, the integral is calculated for all pixels along a line perpendicular to the corresponding region of interest, a non-perpendicular line to the corresponding region of interest, a contour along the corresponding region of interest, or a surface of the corresponding region of interest, and the integral calculated for all pixels is assigned as the pixel value for the temporally corresponding portion of the representative line through the region of interest along the y-axis. In some cases, selecting the corresponding region of interest from the target area across the set of temporal difference images is received via manual input. In some cases, selecting the corresponding region of interest from the target area across the set of temporal difference images is performed by automatic anatomical object recognition.

In some cases, wherein the regional motion display further comprises one or more time-synchronization elements displayed along the x-axis. In some cases, the target area is a heart of a patient and the one or more time-synchronization elements is a temporally corresponding electrocardiogram displayed along the x-axis. In some cases, the temporal set of images of the target area includes at least 250 images per second.

In some cases, the method further includes adjusting the regional motion display by temporal scaling. In some cases, the method further includes automatically detecting a propagating event. In some cases, the method further includes measuring at least one of onset timing of the propagating event, duration of the propagating event, and velocity of the propagating event.

A computing system providing a regional motion display includes a processor, memory, and instructions stored in the memory that when executed by the processor, direct the computing device to perform the method described above.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter.

### BRIEF DESCRIPTION OF THE DRAWINGS

FIG. 1 illustrates a flowchart for providing a regional motion display.

FIGS. 2A and 2B illustrate an acquired B-mode images in an apical four-chamber view.

FIG. 2C illustrates a difference image generated from FIGS. 2A and 2B.

FIG. 2D illustrates a composite of the spatial locations of a propagating event from a sequence of difference images.

FIG. 3 illustrates a selection of a corresponding region of interest from a difference image.

FIGS. 4A-4D illustrate regional motion displays.

FIG. 5 illustrates a computing system that can be used for providing a regional motion display.

FIG. 6 illustrates a specific embodiment of a flowchart for providing a regional motion display.

FIG. 7 illustrates a plot of the time intervals between the LDPE and ESPE, ESPE and LSPE, and of LSPE and MDPE against the R-R interval for all participants in an experiment.

FIG. 8 illustrates a plot of the MDPE-to-LDPE interval against the R-R for all participants in an experiment.

### DETAILED DESCRIPTION

Regional motion display and assessment displays are provided. Advantageously, movement of tissue and/or materials over time is displayed in an easily consumable format. When applied to certain areas of a living entity (e.g., a heart of a human), detailed information including movement of tissue (e.g., a propagating event) over a period of time (e.g., a myocardial tissue over a cardiac cycle) is presented in a format such that physicians can identify abnormalities and disease in a relatively short period of time compared to conventional diagnostic methods. When applied to non-living materials, the movement of the materials is presented in a format such that abnormalities and defects that are otherwise undetectable are readily identifiable.

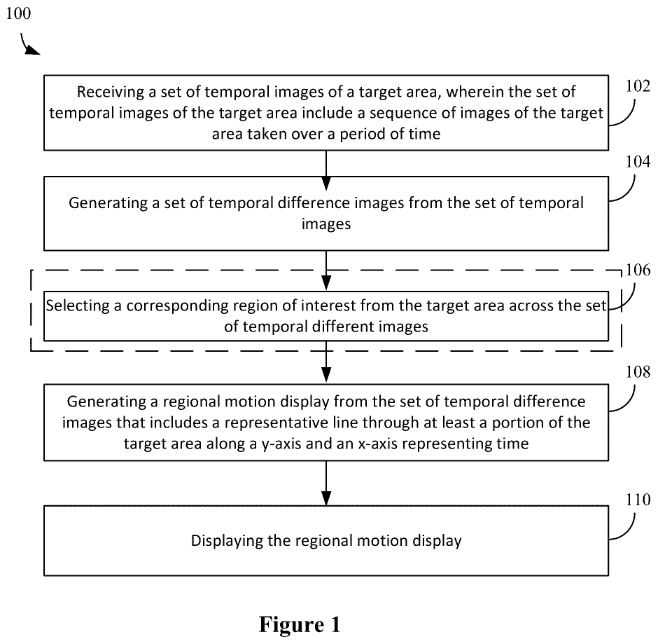

FIG. 1 illustrates a flowchart for providing a regional motion display. Referring to FIG. 1, the method 100 includes receiving (102) a set of temporal images of a target area. The set of temporal images of the target area include a sequence of images of the target area taken over a period of time. In some cases, the set of temporal images is received (102) instantaneously and/or continuously from an imaging system that captures the set of temporal images. In some cases, the set of temporal images is received (102) from storage some time after the set of temporal images are captured via an imaging system. The method 100 further includes generating (104) a set of temporal difference images from the set of temporal images.

In some cases, a difference image for two temporally sequential images (e.g., of the target area) is generated (104) by calculating an absolute difference in detected brightness value of each pixel in the two temporally sequential images and assigning the absolute difference in the detected brightness value as a brightness value of a corresponding pixel in a difference image of the set of temporal difference image. This process can be repeated to generate (104) a difference image between any/all two temporally sequential images.

For example, if there are five temporally sequential images in a set of temporal images of a target area (e.g., image A, image B, image C, image D, and image E), a set of temporal difference images is generated (104) by calculating an absolute difference in detected brightness value of each pixel between image A and image B, between image B and image C, between image C and image D, and between image D and image E. Then, the absolute difference in the detected brightness between image A and image B is assigned as a brightness value of a corresponding pixel in a first difference image, the absolute difference in the detected brightness between image B and image C is assigned as a brightness value of a corresponding pixel in a second difference image, the absolute difference in the detected brightness between image C and image D is assigned as a brightness value of a corresponding pixel in a third difference image, and the absolute difference in the detected brightness between image D and image E is assigned as a brightness value of a corresponding pixel in a fourth difference image. Therefore, assuming a difference image is generated (104) between each two temporally sequential images that are received (102), there would be one less difference image than the total number of temporal images of the set of temporal images of the target area that are received (102). For this example, there would be four difference images (e.g., first difference image, second difference image, third difference image, and fourth difference image) generated (104) in the set of temporal difference images from the set of temporal images. It should be understood that this example is used for illustrative purposes and there may be many more temporally sequential images that are received (102) in the set of temporal images of the target area and generated (104) in the set of temporal difference images.

In some cases, generating (104) the set of temporal difference images from the set of temporal images includes, for two temporally sequential images of the set of temporal images, calculating an absolute difference in detected brightness value of each pixel, applying a brightness transfer function to the absolute difference in the detected brightness value of each pixel, and assigning a result of the brightness transfer function as a brightness value of a corresponding pixel in a difference image of the set of temporal difference images.

In some cases, the method 100 includes selecting (106) a corresponding region of interest from the target area across the set of temporal images or temporal difference images. For example, if the target area is a heart of a patient, the corresponding region of interest that is selected (106) could be all or a portion of the anatomy of the heart. In this way, specific portions of the target area that are of interest may be selected for generation of the regional motion display (e.g., as explained in detail below) from images that include a target area that is larger than that corresponding region of interest. This can be useful for a variety of reasons, including but not limited to reducing necessary processing power needed to perform the method 100, use of a set of temporal images that may have been taken for other and/or dual purposes, and highlighting the corresponding region of interest for a physician and/or other qualified person to use for medical and/or material diagnosis. In some cases, the selecting (106) of a corresponding region of interest from the target area may be done after receiving (102) the set of temporal images of the target area and before the generating (104) of the set of temporal difference images from the set of temporal images.

In some cases, the selecting (106) of the corresponding region of interest from the target area is received via manual input. For example, a user (e.g., physician and/or technician) may manually select the corresponding region of interest by drawing a box and/or shape around the corresponding region of interest via a computer input device (e.g., a mouse, touch screen, keyboard, microphone, or the like). In some cases, the selecting (106) of the corresponding region of interest from the target area is performed by automatic anatomical object recognition. For example, an object recognition imaging program may be designed to identify objects (e.g., a left ventricle of a heart) from images of a target area (e.g., a heart) based on predetermined instructions.

The method 100 further includes generating (108) a regional motion display from the set of temporal difference images. The regional motion display includes a representative line through the target area along a y-axis and an x-axis representing time. In some cases, generating (108) the regional motion display from the set of temporal difference images includes calculating, for each y-axis pixel of the regional motion display, an integral of all pixels, where pixel brightness may be weighted in amplitude, along a line perpendicular to the target area, a non-perpendicular line to the target area, a contour along the target area, or a surface of the target area in a temporally corresponding difference image of the set of temporal difference images, and assigning, for each y-axis pixel of the regional motion display, the integral of all pixels along the line perpendicular to the target area, the non-perpendicular line to the target area, the contour along the target area, or the surface of the target area as a pixel value for a temporally corresponding portion of the representative line through the target area along the y-axis. In some cases, generating (108) the regional motion display includes a brightness transfer function that maps the absolute brightness values to another brightness and/or color scheme. For example, generating (108) the regional motion display from the set of temporal difference images further comprises applying, for each y-axis pixel of the regional motion display, a brightness transfer function to the integral of all pixels along the line perpendicular to the target area, the non-perpendicular line to the target area, the contour along the target area, or the surface of the target area, and a result of the brightness transfer function is assigned as the pixel value for the temporally corresponding portion of the representative line through the target area along the y-axis

In cases that include selecting (106) a corresponding region of interest from the target area across the set of temporal images or temporal difference images, the integral is calculated for all pixels along a line perpendicular to the corresponding region of interest, the non-perpendicular line to the corresponding region of interest, the contour along the corresponding region of interest, or the surface of the corresponding region of interest and the integral calculated for all pixels is assigned as the pixel value for the temporally corresponding portion of the representative line through the corresponding region of interest along the y-axis.

The method 100 further includes displaying (110) the regional motion display. For example, the regional motion display can be displayed on a display screen, printed, produced by a chart recorder, or a x-y recorder. In other examples, the regional motion display can be sent to another device (e.g., via wired or wireless communication means) for displaying on that device.

In some cases, the method 100 further includes adjusting the regional motion display by temporal scaling and/or record length. In some cases, temporal scaling and/or record length of the regional motion display is accomplished by inputting a desired time frame and/or group of the plurality of temporal difference images (e.g., that correspond to a time frame) into a graphical user interface. In some cases, temporal scaling and/or record length of the regional motion display is accomplished by selecting a desired area around within the regional motion display (e.g., selecting a box around a desired portion of the regional motion display for adjustment via temporal scaling).

In some cases, the method 100 further includes automatically detecting a propagating event (e.g., via detection software). In some cases, features of the propagating event are automatically measured. These features can include, but are not limited to, onset timing of the propagating event, duration of the propagating event, and velocity of the propagating event.

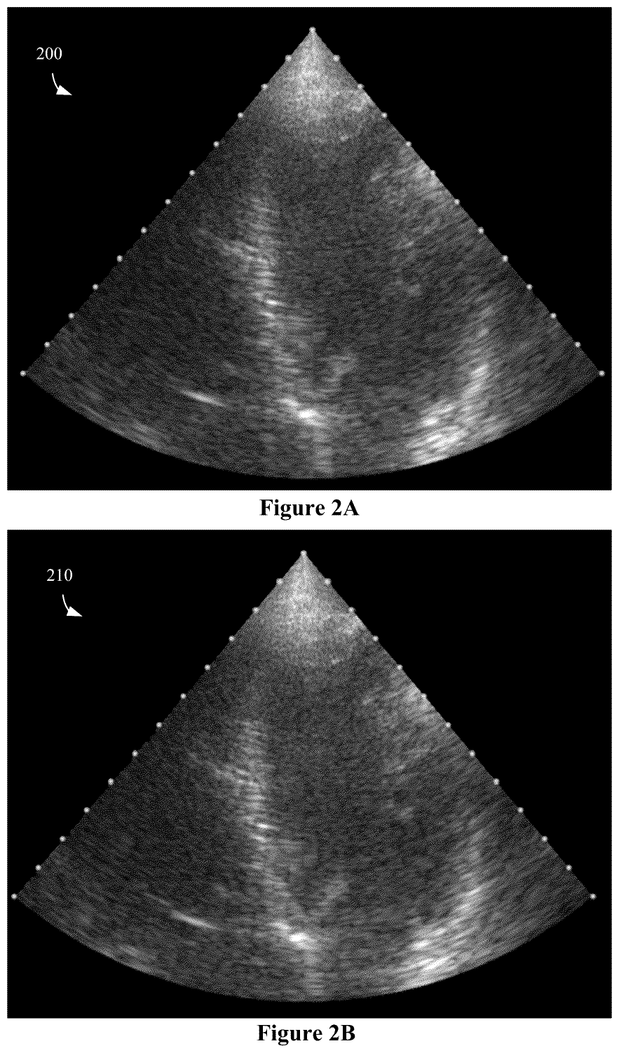

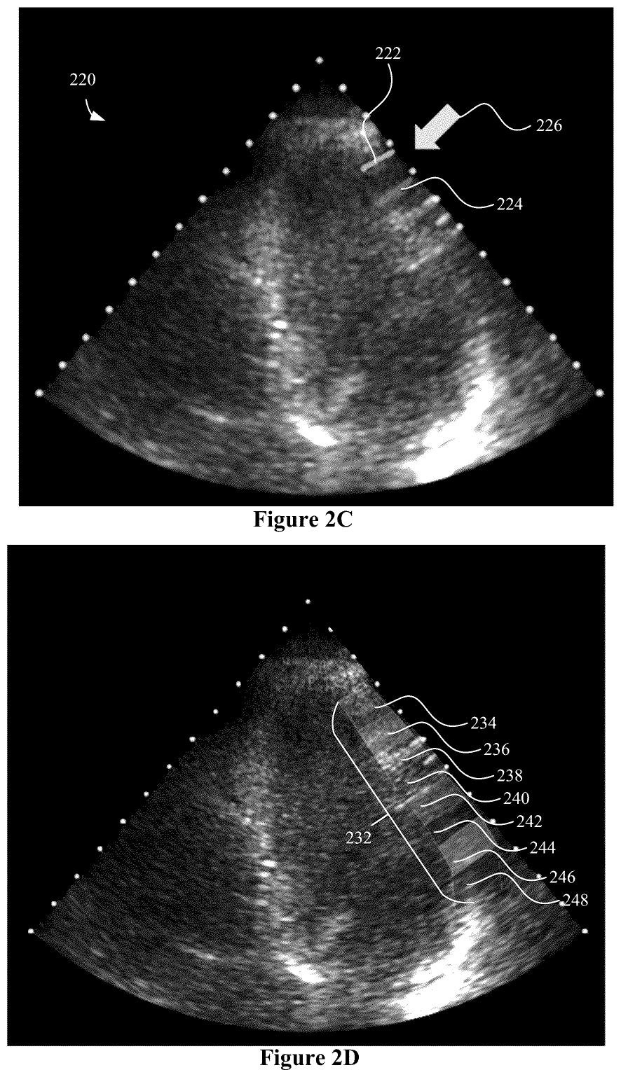

FIGS. 2A and 2B illustrate an acquired B-mode images in an apical four-chamber view. Referring to FIG. 2A, a first image 200 of the apical four-chamber view (e.g., the target area) of a heart of a twenty-five year old male patient is illustrated. Referring to FIG. 2B, a second image 210, captured at a different time, of the apical four-chamber view of the heart of the same twenty-five year old male patient is illustrated. These images may be received by a regional motion assessment imaging device that is received as part of a set of temporal images of a target area as described above with the receiving (102) step of the method 100 described above.

Referring to FIGS. 2A and 2B, the images 200, 210 of the apical four-chamber view of the heart are captured from late diastole. The second image 210 is acquired and/or captured two milliseconds after the first image 200 via an imaging system. In some cases, images of a target area are captured at a rate of 250 images or more per second via an imaging system. In some cases, images of a target area are acquired and/or captured at a rate of 300 images or more per second via an imaging system. In some cases, images of a target area are acquired and/or captured at a rate of 400 images or more per second via an imaging system. In some cases, images of a target area are acquired and/or captured at a rate of 500 images or more per second via an imaging system. In other applications, imaging rates may be higher or lower. In some cases, other types of images (e.g., 2D and/or 3D) images may be used as the set of temporal images.

FIG. 2C illustrates a difference image generated from FIGS. 2A and 2B. Referring to FIG. 2C, a difference image 220 is generated from the first image 200 and the second image 210. This generation of the difference image 220 may occur, for example, as part of the same process as described with respect to generation (104) of a set of temporal difference images described with respect to the method 100. In some cases, the difference image 220 is generated by calculating an absolute difference in detected brightness value of each pixel in the first image 200 and the second image 210. The absolute difference in the detected brightness value is then assigned as a brightness value of a corresponding pixel in the difference image 220. An apical edge 222 of a propagating event is indicated by a first line and a basal edge 224 of the propagating event is indicated by a second line.

As described herein, a propagating event refers to a wave and/or vibration through an object and/or a lack of movement of an object during a wave and/or vibration through that object. For example, a propagating event includes a wave and/or vibration (and/or the lack of movement before, during, and/or after that wave and/or vibration) that occurs along a lateral wall in an apical four-chamber view of a heart of a patient. Other examples include propagating events that occur in other tissue in human patients and/or animals. Still even further examples include propagating events that occur in inanimate synthetic, and/or artificial objects. These propagating events may be induced (e.g., the ringing of a bell), naturally occurring (e.g., via the pumping of a heart), and/or a product of that object (e.g., earth tremors).

FIG. 2D illustrates a composite of the spatial locations of a propagating event from a sequence of difference images. Referring to FIG. 2D, the position and spatial extent of the propagating event 232 at first appearance is indicated by 234. In the next difference image that is shown as part of the composite, the propagating event 232 has moved to the location indicated by 236, 2 milliseconds later. The continued progression of the propagating event 232 indicated by locations 238, 240, 242, 244, 246, and 248 indicate the continued progression of the propagating event through the composite in 2 millisecond increments, representing the total spatial course of the propagating event over the 14 millisecond time period. This represents a propagation velocity of approximately 5 m/sec.

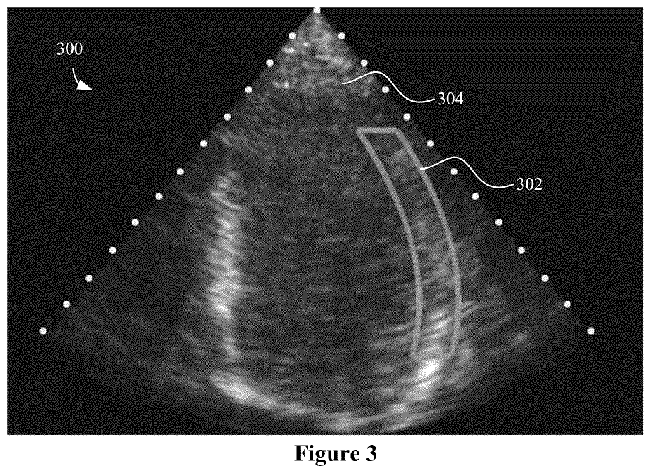

FIG. 3 illustrates a selection of a corresponding region of interest from a difference image. Referring to FIG. 3, a corresponding region of interest 302 is selected from a target area 304 of a difference image 300. It should be understood that, in some cases, the selection of the corresponding region of interest 302 from the target area 304 may occur prior to generation of the difference image 300 (and/or set of temporal difference images) from a received temporal image (and/or set of temporal images of a target area). Furthermore, the selection of the corresponding region of interest 302 from the target area 304 may occur, for example, as part of the same process as described with respect to selection (106) of a corresponding region of interest described with respect to the method 100.

In some cases, selection of the corresponding region of interest 302 from the target area 304 is received via manual input. In some cases, the selection of the corresponding region of interest 302 from the target area 304 is performed by automatic anatomical object recognition.

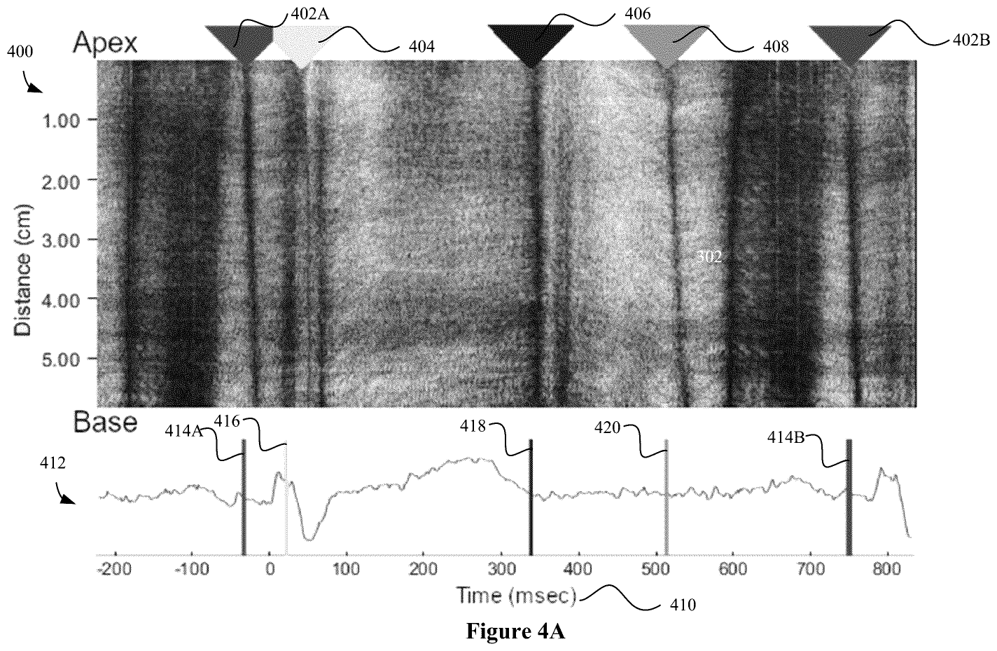

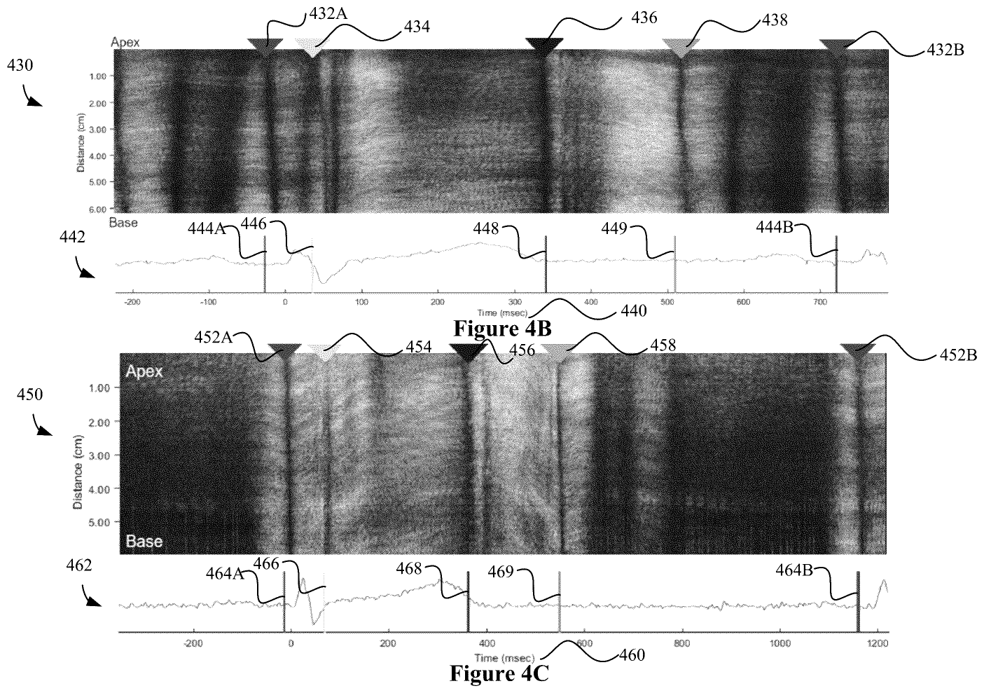

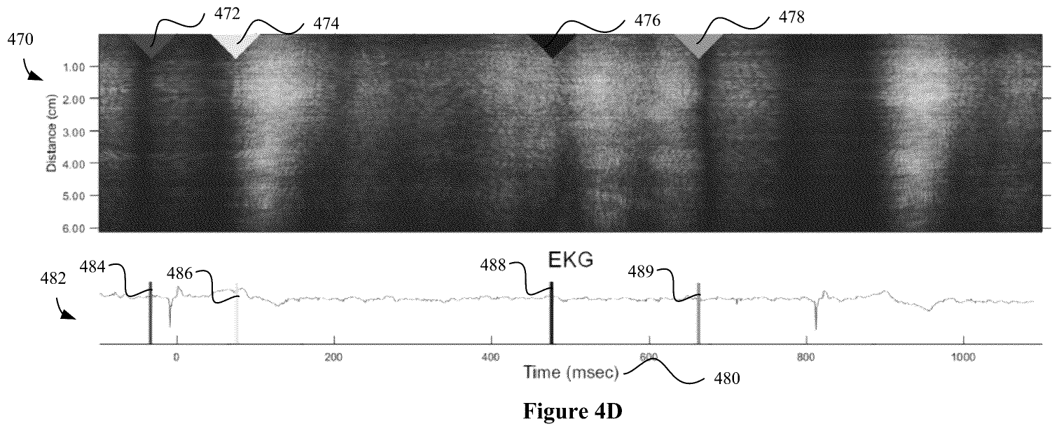

FIGS. 4A-4D illustrate regional motion displays. These regional motion displays illustrate motion throughout a cardiac cycle along a lateral wall from the apical four-chamber views. These regional motion displays are each generated, for example, as part of the same process as described with respect to generation (108) from a set of temporal difference images described with respect to the method 100.

Referring to FIG. 4A, a regional motion display 400 illustrates a plurality of propagating events 402A, 404, 406, 408, and 402B through a target area (or, in some cases, a corresponding region of interest from the target area) of a difference image (e.g., difference images 2C and/or 2D). The regional motion display 400 includes a representative line through the target area (e.g., or corresponding region of interest) along a y-axis and an x-axis that represents time 410 (e.g., in milliseconds). The regional motion display 400 is generated from a set of temporal difference images. For example, each propagating event 402A, 404, 406, 408, and 402B represents a plurality of the position and timing of the propagating events in the cardiac cycle displayed in an easily consumable format in the regional motion display 400. For example, 402A corresponds to the position and timing of the propagating event 232 in FIG. 2D.

The regional motion display 400 is generated from a set of temporal difference images that are generated from a set of temporal images of a heart of a twenty-one year old male patient that are captured at an imaging rate of five-hundred (500) frames per second. In this case, an electrocardiogram (ECG) 412 that is synchronized over time 410 (e.g., along the x-axis) with the regional motion display 400 is also illustrated.

In some cases, a regional motion display may include other time-synchronization elements related to a target area (or corresponding region of interest) in addition to or instead of an electrocardiogram. For example, cycles of machinery, vibration patterns represented by that machinery, and/or other synchronization elements relating to objects (e.g., where those objects represent the target area and/or corresponding region of interest), such as roads and/or bridges, may be displayed along with the regional motion display. In some cases, a regional motion display may include other time-synchronization elements related to a target area (or corresponding region of interest) of a patient.

In some cases, a propagating event (e.g., 402A, 404, 406, 408, and 402B) is automatically detected via detection software. In some cases, features of the propagating event are automatically measured. These features can include, but are not limited to, onset timing of the propagating event, duration of the propagating event, and velocity of the propagating event.

Referring back to FIG. 4A, propagating events 402A and 402B of the regional motion display 400 occur in the late diastolic period of the patient's cardiac cycle, as can be seen from the markers 414A and 414B on the ECG 412. Propagating event 404 of the regional motion display 400 occurs in the early systolic period of the patient's cardiac cycle, as can be seen from the marker 416 on the ECG 412. Propagating event 406 of the regional motion display 400 occurs in the late systolic period of the patient's cardiac cycle, as can be seen from the marker 418 on the ECG 412. Propagating event 408 of the regional motion display 400 occurs in the mid-diastolic period of the patient's cardiac cycle, as can be seen from the marker 420 on the ECG 412.

In some cases, generation of the regional motion display 400 includes calculating, for each y-axis pixel of the regional motion display 400, an integral of all pixels along a line perpendicular to the target area in a temporally corresponding difference image of the set of temporal difference images and assigning, for each y-axis pixel of the regional motion display 400, the integral of all pixels along the line perpendicular to the target area as a pixel value for a temporally (e.g., along the x-axis) corresponding portion of the representative line through the target area along the y-axis. For example, each difference image is represented by a representative line (e.g., along the y-axis) in the regional motion display 400. For each pixel (e.g., along the y-axis) in that representative line of the regional motion display 400, an integral of all pixels along a line perpendicular to the to the target area in the temporally corresponding difference image is calculated. Once the integral is calculated from the line perpendicular to the to the target area in the temporally corresponding difference image for each pixel in that representative line (e.g., along the y-axis) of the regional motion display 400, that integral is assigned a pixel value for the corresponding portion of that representative line in the regional motion display 400.

In some cases in which a corresponding region of interest is selected from the target area across the set of temporal images and/or across the set of temporal difference images, the integral is calculated for all pixels along a line perpendicular to the corresponding region of interest and the integral calculated for all pixels along the line perpendicular to the region of interest is assigned as the pixel value for the temporally corresponding portion of the representative line through the region of interest along the y-axis.

Although this description includes that the integral of all pixels are calculated along a line perpendicular to the target area, in some cases, the integral of all pixels are calculated along a non-perpendicular line target area, a contour along the target area, and/or a surface of the target area. Furthermore, the integral may be calculated using a weighted function across the line, contour, and/or surface of the target area. For example, a triangular weighted function of the line perpendicular to the target area can be used to calculate the integral of all pixels, which would emphasize the pixels in the center of the target area (e.g., cardiac wall) over the pixels at the edges of the target area. In some cases, the weighted function is a spatial weighted function, a temporal weighted function, and/or a spatiotemporal weighted function.

In some cases, the representative line (e.g., through the target area and/or corresponding region of interest of each difference image) that makes up the regional motion display 400 is vertical (e.g., along the y-axis) in length and is one pixel wide (e.g., along the x-axis) for each difference image. In some cases, the representative line that makes up the regional motion display 400 is vertical (e.g., along the y-axis) in length and is two to twenty pixels wide (e.g., along the x-axis) for each difference image. In some cases, the width of each representative varies by the size of the display, number of pixels in a display along the x-direction, the amount of time that is captured across the set of temporal images for display, the number of temporal images received or difference images generated for display and/or the frame rate of the image acquiring system. These variables may be dependent on the application and the characteristics of the object being imaged. Furthermore, the regional motion display 400 can be adjustable by temporal scaling and/or record length. For example, if thirty seconds of data is initially displayed in the regional motion display 400, the regional motion display 400 can be adjusted by temporal scaling and/or record length to view ten seconds of that data, which would enlarge the width of the representative line that represents each difference image (e.g., from two pixels wide to five pixels wide). As another example, if thirty seconds of data is initially displayed in the regional motion display 400, the regional motion display 400 can be adjusted by temporal scaling and/or record length to view sixty seconds of that data, which would reduce the width of the representative line that represents each difference image (e.g., from two pixels wide to one pixel wide). In some cases, temporal scaling and/or record length of the regional motion display is accomplished by inputting a desired time frame and/or group of the plurality of temporal difference images (e.g., that correspond to a time frame) into a graphical user interface. In some cases, temporal scaling and/or record length of the regional motion display is accomplished by selecting a desired area around within the regional motion display (e.g., selecting a box around a desired portion of the regional motion display for adjustment via temporal scaling and/or record length).

The representative line through the target area and/or corresponding region of interest of each difference image is utilized for each difference image. Therefore, the regional motion display 400 includes the same representative line through the target area and/or corresponding region of interest but with varying detected brightness values for each difference image. Therefore, each representative line that makes up each propagating event 402A, 404, 406, 408, and 402B of the regional motion display 400 is generated from a difference image of the set of temporal difference images. In other words, a target area and/or corresponding region of interest of a single difference image is represented in the regional motion display 400 as a vertical line of y-axis pixels (the representative line); and the immediately adjacent vertical line of y-axis pixels (e.g., to the right) in the regional motion display 400 represents the target area and/or corresponding region of interest of the next temporal difference image of the set of temporal difference images. Each pixel of the regional motion display is generated by calculating an integral of all pixels along a line perpendicular (e.g., in the x-axis direction) of representative line through the target area (or, in some cases, the corresponding region of interest from the target area) of a sequentially temporal difference image, with the representative line having a length in y-axis pixels. In this way, movement within the target area (or in some cases, a corresponding region of the target area) is represented across time 410 by the set of temporal difference images. Therefore, the regional motion display 400 may represent and/or be generated from hundreds, thousands, or even tens of thousands of temporal difference images at a given time. In other words, from left to right along the x-axis, the regional motion display 400 is a representation of the entire set of sequentially temporal difference images that are generated from the set of sequentially temporal images that are captured over a period of time 410.

It should be understood that although the regional motion display 400 described in this example is made of the representative line of each difference image that is generated with x-axis and y-axis properties, other regional motion displays may be generated with a representative line having properties of any direction. In some cases, other regional motion displays may be generated with a representative line having properties having properties of any predefined direction and may change from image to image e.g. in the case of tracking.

In some cases, the regional motion display 400 may be instantaneously updated representing real-time updates via captured temporal images of the target area and subsequent generation of temporal difference images that are used to generate the instantaneously updated regional motion display 400 (e.g., producing a scrolling display or a chart recorder). In some cases, the generation of the regional motion display 400 is entirely analog and may not include any storage of the imaging data.

Referring to FIG. 4B, a regional motion display 430 includes a plurality of propagating events 432A, 434, 436, 438, and 432B through a target area (or, in some cases, a corresponding region of interest from the target area) of a difference image along a y-axis and an x-axis that represents time 440 (e.g., in milliseconds). The regional motion display 430 is generated from a set of temporal difference images.

The regional motion display 430 is generated from a set of temporal difference images that are generated from a set of temporal images of a heart of a twenty-one year old male patient that are captured at an imaging rate of one thousand (1000) frames per second. In this case, an electrocardiogram (ECG) 442 that is synchronized over time 440 (e.g., along the x-axis) with the regional motion display 430 is also illustrated. Propagating events 432A and 432B of the regional motion display 430 occur in the late diastolic period of the patient's cardiac cycle, as can be seen from the markers 444A and 444B on the ECG 442. Propagating event 434 of the regional motion display 430 occurs in the early systolic period of the patient's cardiac cycle, as can be seen from the marker 446 on the ECG 442. Propagating event 436 of the regional motion display 430 occurs in the late systolic period of the patient's cardiac cycle, as can be seen from the marker 448 on the ECG 442. Propagating event 438 of the regional motion display 430 occurs in the mid-diastolic period of the patient's cardiac cycle, as can be seen from the marker 449 on the ECG 442.

Referring to FIG. 4C, a regional motion display 450 includes a plurality of propagating events 452A, 454, 456, 458, and 452B through a target area (or, in some cases, a corresponding region of interest from the target area) of a difference image along a y-axis and an x-axis that represents time 460 (e.g., in milliseconds). The regional motion display 450 is generated from a set of temporal difference images.

The regional motion display 450 is generated from a set of temporal difference images that are generated from a set of temporal images of a heart of a nineteen year old male patient that are captured at an imaging rate of five-hundred (500) frames per second. In this case, an electrocardiogram (ECG) 462 that is synchronized over time 460 (e.g., along the x-axis) with the regional motion display 450 is also illustrated. Propagating events 452A and 452B of the regional motion display 450 occur in the late diastolic period of the patient's cardiac cycle, as can be seen from the markers 464A and 464B on the ECG 462. Propagating event 454 of the regional motion display 450 occurs in the early systolic period of the patient's cardiac cycle, as can be seen from the marker 466 on the ECG 462. Propagating event 456 of the regional motion display 450 occurs in the late systolic period of the patient's cardiac cycle, as can be seen from the marker 468 on the ECG 462. Propagating event 458 of the regional motion display 450 occurs in the mid-diastolic period of the patient's cardiac cycle, as can be seen from the marker 469 on the ECG 462.

Referring to FIG. 4D, a regional motion display 470 includes a plurality of propagating events 472, 474, 476, and 478 through a target area (or, in some cases, a corresponding region of interest from the target area) of a difference image along a y-axis and an x-axis that represents time 480 (e.g., in milliseconds). The regional motion display 470 is generated from a set of temporal difference images.

The regional motion display 470 is generated from a set of temporal difference images that are generated from a set of temporal images of a heart of patient with cardiac amyloidosis and an implanted right ventricle pacemaker that are captured at an imaging rate of one thousand (1000) frames per second. In this case, an electrocardiogram (ECG) 482 that is synchronized over time 480 (e.g., along the x-axis) with the regional motion display 470 is also illustrated. Propagating event 472 of the regional motion display 470 occurs in the late diastolic period of the patient's cardiac cycle, as can be seen from the marker 484 on the ECG 482. Propagating event 474 of the regional motion display 470 occurs in the early systolic period of the patient's cardiac cycle, as can be seen from the marker 486 on the ECG 482. Propagating event 476 of the regional motion display 470 occurs in the late systolic period of the patient's cardiac cycle, as can be seen from the marker 488 on the ECG 482. Propagating event 478 of the regional motion display 470 occurs in the mid-diastolic period of the patient's cardiac cycle, as can be seen from the marker 489 on the ECG 482.

FIGS. 4B, 4C, and/or FIG. 4D may also include any feature and/or generated/displayed elements as described with respect to FIG. 4A.

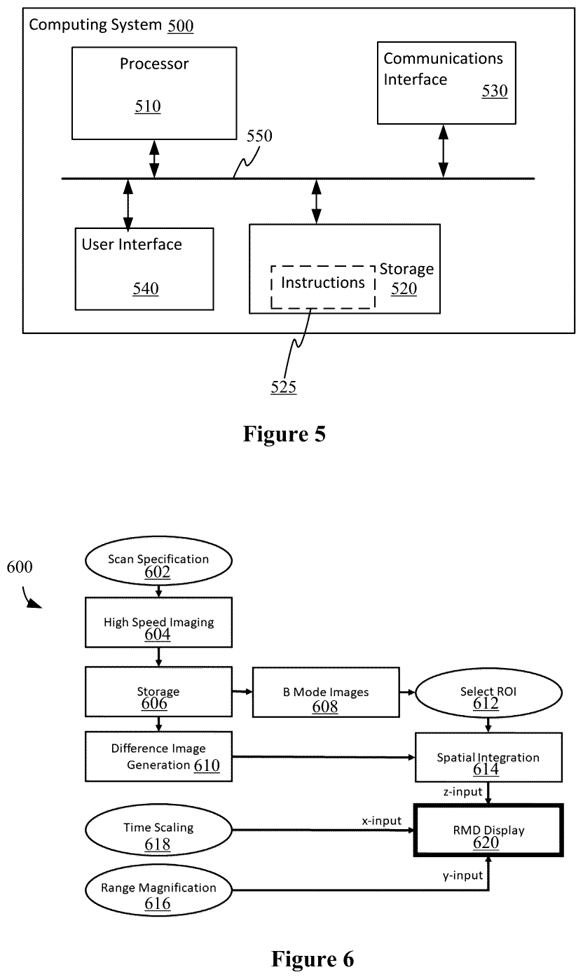

FIG. 5 illustrates a computing system that can be used for providing a regional motion display. Referring to FIG. 5, a computing system 500 can include a processor 510, storage 520, a communications interface 530, and a user interface 540 coupled, for example, via a system bus 550. Processor 510 can include one or more of any suitable processing devices (“processors”), such as a microprocessor, central processing unit (CPU), graphics processing unit (GPU), field programmable gate arrays (FPGAs), application-specific integrated circuits (ASICs), logic circuits, state machines, application-specific standard products (ASSPs), system-on-a-chip systems (SOCs), complex programmable logic devices (CPLDs), etc. Storage 520 can include any suitable storage media that can store instructions 525 for performing any method or process described herein, including method 100 of FIG. 1 and/or any method or process for generating a regional motion display. Suitable storage media for storage 520 include random access memory, read only memory, magnetic disks, optical disks, CDs, DVDs, flash memory, magnetic cassettes, magnetic tape, magnetic disk storage or other magnetic storage devices, or any other suitable storage media. As used herein “storage media” do not consist of transitory, propagating waves. Instead, “storage media” refers to non-transitory media.

Communications interface 530 can include wired or wireless interfaces for communicating with an image acquiring system as well as interfaces for communicating with the “outside world” (e.g., external networks). User interface 540 can include a display on which the regional motion display and any time-synchronization elements can be displayed as well as suitable input device interfaces for receiving user input (e.g., mouse, keyboard, microphone).

Experiment

The inventors conducted an experiment by following a process similar to method 100 described in FIG. 1. Although this experiment is limited to the study of propagating events in the human heart during the cardiac cycle, as explained above, propagating events that are identified and quantified in other tissues, materials, and/or objects using the methods described herein are within the scope of this invention.

In this experiment, the inventors developed a unique method that allows continuous, non-gated data acquisition during normal live, high-speed clinical studies to identify and quantify propagating events. Data analysis uses the detected B-mode brightness values instead of RF data.

This new approach to the identification and quantification of propagating autogenic myocardial events is based on analyzing the motion visualized in difference images (DIs) (e.g., generated from B Mode images) obtained at rates of 500-1000 images per second when played back at slower speeds. Propagating events are not seen in the standard clinical 2-D B-mode images at these high frame rates even when played back at lower speeds, but are reliably visualized with DIs in the left ventricle in apical four-chamber views. Onset timing of propagating events (PEs) was accomplished via reference to simultaneously sampled and displayed synchronous electrocardiograms (ECGs). Initial patient studies using normal participants revealed that this method allows for PE velocity determination, particularly at end diastole. Encouraged by these findings, the inventors developed a new display technique using spatial averaging of difference data in the myocardial walls and displayed the results as a function of range and time. The new display format, regional motion display (RMD), reminiscent of an M-mode display, allows an intuitive appreciation of myocardial wall motion throughout the cardiac cycle and permits timing measurements of PEs with respect to the ECG and velocity determinations. Here the inventors present difference imaging during live clinical scanning, the development of the RMD method, with the initial observations and results from a limited number of clinical participants.

Difference Imaging

For 2-D clinical scans, up to 30 seconds of successive images (e.g., the set of temporal images) are stored with a synchronously acquired ECG signal sampled at the current frame rate (although more or less time for images is contemplated herein). During live, non-gated scanning conventional 2-D B-mode and 2-D DIs can be displayed side by side or separately at the discretion of the operator. During playback, the same display features are available at full or variable slow motion playback speeds. For the current studies, either 64 or 96-element linear arrays are employed operating at 3.5 MHz center frequency. The T5 scanner allows software control of the number of active transmit and receive channels to limit the system noise introduced by unused channels.

Detected B-mode data was stored in its native ρ-θ format, and a standard B-mode image was formed by scan converting these detected brightness values into an image. Difference images were created by subtracting the detected brightness values of two sequential HFR ultrasound images on a sample-by-sample basis and storing the absolute value of the Difference Brightness (DB) such that:

DB_{i,ρ,θ}=|B_{i,ρ,θ}−B_{i-1,ρ,θ}|

where B_{i,ρ,θ} is the brightness value from image i at spatial location (ρ,θ), and DB is the corresponding brightness in the DI. Adjustments for brightness and contrast are independent and under operator control for the two simultaneously displayed B-mode and difference images. The time separation of frames to be subtracted can be varied, and temporal summation and degree of normalization of subtracted images are at the operator's discretion. For all studies presented here, only successive images at frame rate intervals are subtracted and no temporal summation was employed.

Regional Motion Display

The process the inventors used to generate an RMD is outlined in the block diagram in FIG. 6. In the figure, ovals indicate the need for operator interaction while rectangles indicate machine processing of the data. The process 600 includes, at the beginning of a clinical scan, the operator selecting (602) scan parameters such as maximum range, field of view and degree of parallelism, all of which will determine the scan frame rate. The operator then scans (604) the patient indicated by the high-speed imaging block. The echo data from the patient were continuously stored (606) in Storage and B-mode images (608) were presented simultaneously with generated DIs (610) on a regular monitor screen. Once the operator was satisfied that an apical four-chamber view has been visualized, the scan was halted. At that point, the last 20 seconds of scan data was available in the continuously updated storage of the T5 scanner. The operator then selected one cardiac cycle form the stored images to be processed. Then the operator selected the particular cardiac wall to be analyzed from the image coincident with the P-wave and drew (612) the ROI about that wall. Once the ROI was specified, the program calculated (614) the integral of all pixels along a line perpendicular to the drawn contour of the ROI (e.g., the representative line). This repeated for each pixel along the drawn contour. This integrated value is the brightness input, z, to the RMD display. The operator then selected (616) the range magnification along the ROI, the y-axis of the display and the length of the RMD record by selecting (618) x-axis time base. Once all of the parameters are set, the RMD was displayed (620) starting at the P-wave of the selected cardiac cycle. Of course, more than one cardiac cycle or a fraction thereof may be selected by the time scaling. The RMD is a representation of the value of the spatially integrated DI value along the length of the drawn contour as a function of time for the operator selectable time period. For the system used by the inventors, an RMD could be generated for all of the 20 seconds of stored image data. This allowed the capture of irregular heartbeats such as premature ventricular contractions (PVCs).

The velocity of a PE was measured from the RMD by manually tracing the leading edge, that is, the bright to dark transition seen in the RMD, along the wall. The average slope of this line was converted to a velocity. To verify the effectiveness of detecting the late diastolic propagating event (LDPE) and the ability to quantitate the timing and propagation velocity of this event, the inventors compared the frame counted results with the RMD determinations. To assess the effect of frame rate, the inventors studied several participants at 500 and 1000 frames per second during a single study session. RMD images were compared for similarity of appearance, timing and PE velocity determinations using the same RMD horizontal time display rates.

One adult with cardiac amyloidosis was imaged at rates of 500 and 1000 per second on the T5 scanner. This participant had an implanted RV pacemaker. An RMD was generated from the lateral wall of this participant at 1000 images/s.

Results

Clinical Application

Two high-frame-rate images used to generate a DI are provided in FIGS. 2A and 2B. These images were acquired at 500/s and were chosen from late diastole. The resultant DI generated from FIGS. 2A and 2B is provided in FIG. 2C, with the PE appearing near the apex of the lateral wall. These apical four-chamber images were obtained in a 21-y-old volunteer at 3.5 MHz with a 64-element linear array (GE 3Sc, GE Healthcare, Chicago, IL, USA) at 500/s. The PE of interest is seen in the DI as a finite dark area in the lateral wall of the apex indicated by an arrow 226. This LDPE appears to arise at the apex of the heart and quickly travels to the base of the heart. In the DI, motion of tissues produces a brightness amplitude while lack of motion results in a cancellation of the echo signals, thus creating a dark region. By analyzing the time interval between the indicated marker of the PE and the onset of the QRS complex by counting frames, the inventors determined that the PE began 43 ms before the Q-wave, at the end of diastole, before ventricular depolarization and the beginning of isovolumic contraction.

This frame counting process for velocity determination is illustrated in FIG. 2D. In FIG. 2D, the position of the PE in time is indicated by a series of difference image zones 234, 236, 238, 240, 242, 244, 246, and 248 from successive images as the dark area of FIG. 2C propagates down the free wall. These difference image zones 234, 236, 238, 240, 242, 244, 246, and 248 are superimposed on the image in FIG. 2C (e.g., as illustrated in FIG. 2D) for the sake of clarity. Each difference image zone 234, 236, 238, 240, 242, 244, 246, and 248 indicates the position of the PE separated by 1/FR s, which is 2 ms in this example. Using this simple approach, the inventors studied 16 patients, aged 18 to 85, with no prior cardiac diagnosis on standard echo to determine propagation velocities at end diastole.

FIGS. 4A-4D represent regional motion displays that were generated by this experiment. Note that both the leading and trailing edges of the PEs in these figures are slanted in the RMD, indicating propagation from apex to base of the heart. Measurement of the slope of these edges allows for a velocity determination. Table 1 compares the timing of the LDPE event onset as measured by frame counting (T-Fr Ct) with the timing derived from the RMD (T-RMD). LDPE timing is with respect to the onset of the Q-wave in the ECG. Age and heart rate are reported for all 16 participants.

TABLE 1
Velocity of LDPE and timing of four unique propagating
events with respect to the onset of the QRS complex

| | Heart rate | v-FrCt | v-RMD | T-FrCt | T-RMD | T-ESPE | T-LSPE | T-MDPE |
|---|---|---|---|---|---|---|---|---|
| Age | (bpm) | (m/s) | (m/s) | (ms) | (ms) | (ms) | (ms) | (ms) |
| 21 | 77 | 4.5 | 4.8 | −55 | −59 | 41 | 334 | 512 |
| 22 | 62 | 3.4 | 3.25 | −45 | −48 | 22 | 396 | 545 |
| 20 | 55 | 2.7 | 2.51 | −32 | −30 | 43 | 376 | 535 |
| 20 | 56 | 4.1 | 4.3 | −5 | −6 | 73 | 384 | 570 |
| 19 | 47 | 3.2 | 3.21 | −25 | −24 | 63 | 358 | 537 |
| 21 | 78 | 6.4 | 6.29 | −5 | −2 | 39 | 346 | 521 |
| 25 | 62 | 2.5 | 2.43 | −39 | −35 | 46 | 348 | 534 |
| 85 | 62 | 2.56 | 2.38 | −75 | −79 | | | |
| 68 | 95 | 3.8 | 3.13 | −44 | −31 | 78 | 325 | 466 |
| 58 | 70 | 2.2 | 3.22 | −70 | −65 | | 401 | 581 |
| 65 | 82 | 1.97 | 3.54 | −31 | −35 | 36 | 375 | 563 |
| 80 | 61 | 2.04 | 2.54 | −21.4 | −18 | 59 | 417 | 722 |
| 18 | 54 | 2.56 | 4.66 | −29.3 | −28 | 68 | 408 | 654 |
| 49 | 59 | 2.05 | 2.49 | −31.5 | −17 | 27 | 416 | 598 |
| 71 | 64 | 2.79 | 2.03 | −21.5 | −10 | 19 | 395 | 631 |
| 32 | 60 | 2.37 | 2.9 | −55 | −20 | 81 | 391 | 632 |
| Mean | 65 | 3.1 | 3.4 | −34 | −32 | 61 | 380 | 579 |
| SD | | | | | 20.9 | 20.0 | 28.6 | 62.9 |

The LPDE was found to start 34.1 milliseconds before the onset of the QRS complex on average by frame counting. The LPDE found to start 31.7 milliseconds before the onset of the QRS complex on average using the RMD method, with an average difference of 2.4 plus or minus 5.9 milliseconds. Of note, in all volunteers, the LDPE started before the QRS complex, indicating that the origin of this particular PE is not ventricular systole. In all volunteers, the LDPE was observed to propagate from apex to base in the reduced playback speed difference images. The LDPE was measured to have a negative slope in all RMD images, which corresponds to apex-to-base propagation. Velocities of the LDPE as measured from the RMD averaged 3.4 m/s. By frame counting, velocities were measured to be 3.1 m/s on average. The difference between measured velocity by the two methods was 0.3±0.7 m/s.

In some participants, the inventors were able to obtain RMDs derived from 1000 frames per second and compared those derived at 500 frames per second in terms of velocity resolution and accuracy of timing. An example of this is illustrated in FIGS. 4A and 4B. Note the similarity of the RMDs in terms of the temporal location of the PEs, while the reduced visibility of small structures in the diffuse areas is owing to increased temporal sampling, as brightness in DIs is proportional to target velocity and inversely proportional to frame rate.

All participants showed the three other PEs both in the slowed difference images and in the RMDs with consistent timing. However, the pattern varied from participant to participant. The onset of the early systolic propagating event (ESPE) occurring during rapid LV contraction is illustrated in FIGS. 4A-4D. The onset of the late systolic propagating event (LSPE) is illustrated in FIGS. 4A-4D, and is potentially associated with isovolumic relaxation. The mid-diastole (MDPE) may be associated with the elastic rebound of the myocardial lateral wall after rapid filling. Careful inspection of FIG. 4A reveals that the background targets in the RMD appear to move from apex to base before the onset of the ESPE and change direction to move toward the apex after the ESPE.

The onset of the ESPE occurs 46 ms after the onset of the QRS on average and was observed to be a series of alternating bright and dark bands. In the participants included in this study, this event was seen as two to four bright peaks separated by one to three dark bands. A slope, and thus a velocity, could not be reproducibly measured. The next event, the LSPE, occurs on average 365 ms after the QRS and is characterized by one or two dark bands in the RMD. Like the ESPE, these dark bands are nearly vertical, making it difficult to reproducibly measure a velocity. The steep or vertical slope could indicate a velocity higher than the temporal resolution of this method or could arise from out-of-plane propagation.

The fourth event, the MDPE, occurs 536 milliseconds after the QRS on average. This event presents as a single dark band that separates the brighter phase of early diastole and the dimmer phase of late diastole. This event has a measurable sloped edge; however, the velocity and direction varied among participants. All four events were found consistently timed to the onset of the QRS across three or more cardiac cycles in each participant and in all participants regardless of age or heart rate. The timing data of the ESPE, LSPE and MDPE are tabulated in Table 1. Some entries are missing because of poor original DI quality. The data presented represent an example of measurements made possible with this new display method.

These PEs are probably guided shear waves or pulses propagating in a shear mode initiated by hemodynamic transients and their momentum transfer to the myocardial walls. The lack of signal during the PE in DIs indicates that there is no detectable target or speckle motion during the event. The propagating events illustrated in FIGS. 4A-4D are all associated with contractile events. Noteworthy is the observation that in all 16 participants, the four main PEs, that is, the LDPE, ESPE, LSPE and MDPE, were repeated at the same interval between cardiac cycles as the R-R interval with a slope of 1 and R² of 0.97.

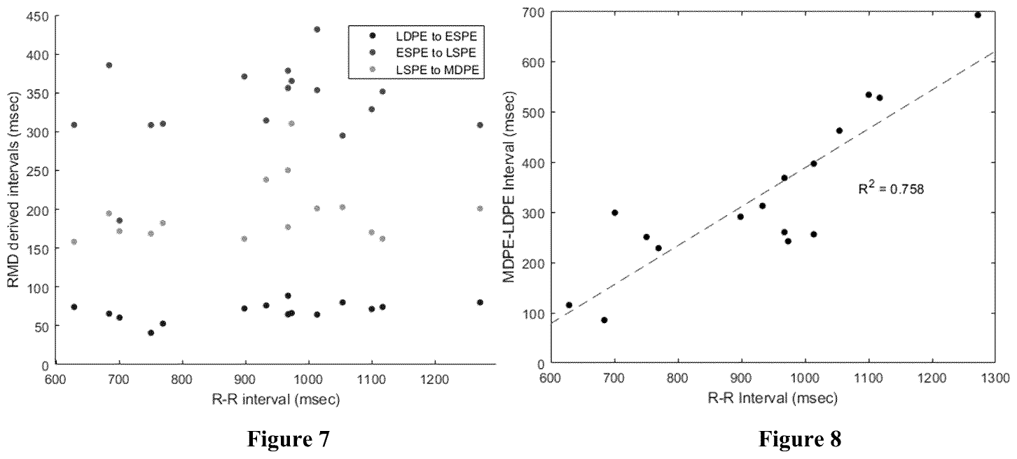

FIG. 7 plots the time intervals between the LDPE and ESPE, ESPE and LSPE, and of LSPE and MDPE versus the R-R interval for all participants. Past studies have cited a linear relationship between ejection time and heart rate. These results indicate that the LDPE-to-ESPE interval is independent of heart rate. The LSPE-to-MDPE interval indicates more variation; however, this variation does not appear to be strongly correlated to heart rate. The ESPE-to-LSPE interval has more variation that is weakly correlated to heart rate; however, none of these are statistically significant. This method 600 provides a new way to divide systolic intervals based on the discrete PEs seen in the myocardium. With this approach, the phases of systole can be identified with higher temporal resolution independent of other observations such as valve closure and synchronous ECG.

On the other hand, the MDPE-to-LDPE interval is directly related to diastole as illustrated in FIG. 8. That interval is plotted against the R-R interval for the 16 participants. The linear fit is approximate, with an R² of 0.76.

An interesting observation on the individual RMDs of participants is that the PE line structure varies among participants. For example, the RMD of FIG. 4A reveals an ESPE that contains only one dark band, unlike the RMD in FIG. 4C, which reveals dark bands.

Participant with Cardiac Amyloidosis

The lateral wall RMD and synchronous ECG from the participant with cardiac amyloidosis can be seen in FIG. 4D. Referring back to FIG. 4D, the RMD 470 is generated from images acquired at 1000/s and is temporally segmented slightly differently from that of the normal participants. The RMD 470 was started before the P-wave in a cardiac cycle with normal P-wave presentation and continued through the end of active systolic contraction on the next beat. For consistency, the onset of the QRS complex in the ECG 482 was used as the zero time, and the ventricular pacing spike as measured from the ECG 482 occurred at −52 milliseconds in both beats. The LPDE 472 occurred at −30 ms, the ESPE 474 occurred at 77 milliseconds, the LSPE 476 occurred at 478 milliseconds, and the MPDE 478 occurred at 665 milliseconds. All four propagating events 472, 474, 476, and 478 occurred later than the average normal cohort but were within two standard deviations of the normal cohort averages (see Table 1). As the appearance of the four propagating events 472, 474, 476, and 478 in the participant with amyloidosis were different from those in the normal cohort, the onset of each propagating event 472, 474, 476, and 478 was marked in a fashion consistent with the normal participants. That is, at the leading edge of the event at the bright-to-dark transition. Of note, in the second cardiac cycle of FIG. 4D, the ventricular pacing spike occurred before the natural atrial contraction, and the LDPE 472 is not visualized in the RMD 470 across the full lateral wall. The edge of the propagating event 472 appears to have a slope indicating propagation from apex to base, with velocity measured to be 5.3 m/s.

Although the subject matter has been described in language specific to structural features and/or acts, it is to be understood that the subject matter defined in the appended claims is not necessarily limited to the specific features or acts described above. Rather, the specific features and acts described above are disclosed as examples of implementing the claims and other equivalent features and acts are intended to be within the scope of the claims.

What is claimed is:

1. A method comprising:
receiving a set of temporal images of a target area, wherein the set of temporal images of the target area comprise a sequence of images of the target area taken over a period of time;
generating a set of temporal difference images from the set of temporal images;
generating a regional motion display from the set of temporal difference images comprising a representative line through the target area along a y-axis and an x-axis representing time; and
displaying the regional motion display.

2. The method of claim 1, wherein generating the set of temporal difference images from the set of temporal images comprises, for two temporally sequential images of the set of temporal images:
calculating an absolute difference in detected brightness value of each pixel; and
assigning the absolute difference in the detected brightness value as a brightness value of a corresponding pixel in a difference image of the set of temporal difference images.

3. The method of claim 1, wherein generating the regional motion display from the set of temporal difference images comprises:
calculating, for each y-axis pixel of the regional motion display, an integral of all pixels along a line perpendicular to the target area, a non-perpendicular line to the target area, a contour along the target area, or a surface of the target area in a temporally corresponding difference image of the set of temporal difference images; and
assigning, for each y-axis pixel of the regional motion display, the integral of all pixels along the line perpendicular to the target area, the non-perpendicular line to the target area, the contour along the target area, or the surface of the target area as a pixel value for a temporally corresponding portion of the representative line through the target area along the y-axis.

4. The method of claim 3, further comprising selecting a corresponding region of interest from the target area across the set of temporal difference images;
wherein the integral is calculated for all pixels along a line perpendicular to the corresponding region of interest, a non-perpendicular line to the corresponding region of interest, a contour along the corresponding region of interest, or a surface of the corresponding region of interest; and
wherein the integral calculated for all pixels along the line perpendicular to the corresponding region of interest, the non-perpendicular line to the corresponding region of interest, the contour along the corresponding region of interest, or the surface of the corresponding region of interest is assigned as the pixel value for the temporally corresponding portion of the representative line through the corresponding region of interest along the y-axis.

5. The method of claim 4, wherein selecting the corresponding region of interest across the set of temporal difference images is received via manual input.

6. The method of claim 4, wherein selecting the corresponding region of interest across the set of temporal difference images is performed by automatic anatomical object recognition.

7. The method of claim 3, wherein generating the regional motion display from the set of temporal difference images further comprises applying, for each y-axis pixel of the regional motion display, a brightness transfer function to the integral of all pixels along the line perpendicular to the target area, the non-perpendicular line to the target area, the contour along the target area, or the surface of the target area,
wherein a result of the brightness transfer function is assigned as the pixel value for the temporally corresponding portion of the representative line through the target area along the y-axis.

8. The method of claim 3, wherein the integral of all pixels along the line perpendicular to the target area, the non-perpendicular line to the target area, the contour along the target area, or the surface of the target area in a temporally corresponding difference image of the set of temporal difference images is calculated using a weighted function.

9. The method of claim 1, wherein the regional motion display further comprises one or more time-synchronization elements displayed along the x-axis.

10. The method of claim 9, wherein the target area is a heart of a patient, wherein the one or more time-synchronization elements is a temporally corresponding electrocardiogram displayed along the x-axis.

11. The method of claim 1, wherein the set of temporal images of the target area comprises at least 250 images per second.

12. The method of claim 1, further comprising adjusting the regional motion display by temporal scaling.

13. The method of claim 1, further comprising automatically detecting a propagating event.

14. The method of claim 13, further comprising measuring at least one of onset timing of the propagating event, duration of the propagating event, and velocity of the propagating event.

15. A computing device comprising:
a processor, memory and instructions stored in the memory that when executed by the processor, direct the computing device to:
receive a set of temporal images of a target area, wherein the set of temporal images of the target area comprise a sequence of images of the target area taken over a period of time;
generate a set of temporal difference images from the set of temporal images;
generate a regional motion display from the set of temporal difference images comprising a representative line through the target area along a y-axis and an x-axis representing time; and
cause to display the regional motion display.

16. The computing device of claim 15, wherein generating the set of temporal difference images from the set of temporal images comprises, for two temporally sequential images of the set of temporal images:
calculating an absolute difference in detected brightness value of each pixel; and
assigning the absolute difference in the detected brightness value as a brightness value of a corresponding pixel in a difference image of the set of temporal difference images.

17. The computing device of claim 15, wherein generating the regional motion display from the set of temporal difference images comprises:
calculating, for each y-axis pixel of the regional motion display, an integral of all pixels along a line perpendicular to the target area, a non-perpendicular line to the target area, a contour along the target area, or a surface of the target area in a temporally corresponding difference image of the set of temporal difference images; and
assigning, for each y-axis pixel of the regional motion display, the integral of all pixels along the line perpendicular to the target area, the non-perpendicular line to the target area, the contour along the target area, or the surface of the target area as a pixel value for a temporally corresponding portion of the representative line through the target area along the y-axis.

18. The computing device of claim 17, further comprising selecting a corresponding region of interest from the target area across the set of temporal images or temporal difference images;
wherein the integral is calculated for all pixels along a line perpendicular to the corresponding region of interest, a non-perpendicular line to the corresponding region of interest, a contour along the corresponding region of interest, or a surface of the corresponding region of interest; and
wherein the integral calculated for all pixels along the line perpendicular to the corresponding region of interest, the non-perpendicular line to the corresponding region of interest, the contour along the corresponding region of interest, or the surface of the corresponding region of interest is assigned as the pixel value for the temporally corresponding portion of the representative line through the corresponding region of interest along the y-axis.

19. The computing device of claim 18, wherein selecting the corresponding region of interest across the set of temporal difference images is received via manual input.

20. The computing device of claim 18, wherein selecting the corresponding region of interest across the set of temporal difference images is performed by automatic anatomical object recognition.
